(19) United States
(12) Patent Application Publication
(10) Pub. No.: US 2024/0058812 A1
(43) Pub. Date: Feb. 22, 2024

(54) Bio Chip Package Structure

(71) Applicant: HSI-TENG KAO, Hsinchu City (TW)

(72) Inventor: HSI-TENG KAO, Hsinchu City (TW)

(21) Appl. No.: 18/356,143

(22) Filed: Jul. 20, 2023

Related U.S. Application Data
(60) Provisional application No. 63/398,621, filed on Aug. 17, 2022.

(51) Int. Cl. B01L 3/00

(52) U.S. Cl. CPC B01L 3/502707

(57) ABSTRACT

A bio chip package structure is provided, which includes a substrate having a top surface and a bottom surface, and a through hole penetrating the top surface and bottom surface of the substrate; a bio chip having an active surface and a back surface, at least two sides on the active surface of the bio chip having a plurality of I/O pads respectively, and a plurality of biological probes is arranged in area between the plurality of I/O pads, and the active surface of the bio chip is set toward to align the through hole of the substrate; and a mold is arranged on the top surface of the substrate, the mold having an opening to align with the through hole of the substrate, so the plurality of biological probes exposed from the through hole of the substrate and the opening of the mold.

### FIELD OF THE INVENTION

The present invention relates to a technical field of bio sensing, particularly relates to a bio chip package structure.

### BACKGROUND OF THE INVENTION

Biosensors are composed of molecular recognition elements and signal conversion elements, which can convert chemical signals generated by biochemical reactions into electronic physical signals for analysis. The bio chip uses micro-electro-mechanical technology to implant the probe molecules into the bio chip, and then conduct various biochemical analyzes through the biological binding characteristics. The target objects may include genes, protein(s) or cell tissues, and may apply in biomedical research, disease diagnosis, food pathogen detection, environmental analysis and identification, and has the advantages of portability, high analysis sensitivity and specificity, fast analysis speed, and only requires a small amount of detection samples and reagents and is a new field of development in the biotechnology industry.

In the present bio chip package structure, the reaction zone and the electrical connection elements of the bio chip are mostly integrated on the surface of the substrate by wire bonding. For example, the reaction zone of the bio chip is disposed on the surface of the package structure adjacent to the pads and wires. However, in this case, the pads and wires are easily corroded by the strong alkali reaction solution used in bio chip, thereby, the affecting the performance of the bio chip, and the electrical connection elements such as pads and wires arranged on the surface of the substrate may limit the active area of the reaction zone of the bio chip.

### SUMMARY OF THE INVENTION

According to the disadvantages of the conventional prior art, a major object of the present invention is to provide a bio chip package structure. An active surface of the bio chip is faced toward the through hole or window on the bottom surface of the substrate, and the biological probes is exposed out of the through hole or window of the substrate. Accordingly, the wire bonding is not necessary use in electrically connected the bio chip and the substrate, so the volume of the entire bio chip package structure may be narrowed.

Another object of the present invention is to provide a bio chip that is arranged on the bottom surface of the substrate, so it is not to consider that the height difference between the substrate and the bio chip thereon while the mold is used to cover the bio chip and substrate. Accordingly, the mold may fix on the upper surface of the substrate, and there is no gap between the mold and the upper surface of the substrate to allow the water vapor to penetrate into the substrate or leakage of the detection solution results in damage to the entire bio chip package structure.

According to above objects, the present invention provides a bio chip package structure, which includes substrate, a bio chip and a mold. The substrate having a upper surface and a bottom surface, and a through hole passed through the upper surface and the bottom surface of the substrate. The bio chip having an active surface and a back surface. There is a plurality of I/O pads is respectively disposed on two sides of the active surface of the bio chip. There is a plurality of biological probes arranged on a region between the plurality of I/O pads. The active surface of the bio chip is faced up to make the plurality of biological probes that is aligned with the through hole of the substrate. The mold is arranged on the upper surface of the substrate. The mold includes an opening to align with the through hole of the substrate. Accordingly, the through hole of the substrate and the opening of the mold to expose the plurality of biological probes of the bio chip.

### BRIEF DESCRIPTION OF THE DRAWINGS

FIG. 1 is a cross sectional view of showing the structure of bio chip package structure in accordance with the present invention disclosed herein.

FIG. 2 is a schematic diagram of showing the detection of the detection solution by the bio chip package structure in accordance with the present invention disclosed herein.

### DETAILED DESCRIPTION OF THE PREFERRED EMBODIMENT

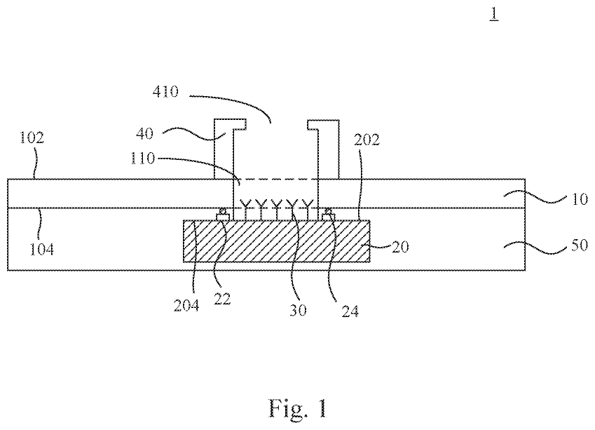

First, please refer to FIG. 1. FIG. 1 is a cross sectional view of showing a bio chip package structure. In FIG. 1, the bio chip package structure 1 is at least composed of a substrate 10, a bio chip 20 and a mold 40. The substrate 10 having a upper surface 102 and a bottom surface 104, and there is a through hole 110 (or may name as window) within the substrate 10 and is passed through the upper surface 102 and the bottom surface 104 of the substrate 10. In some preferred embodiments of this invention, the substrate 10 with the through hole 110 therein may a printed circuit board with one or more layers of circuit layout, and there is a pre-designed circuit layout (now shown) in the substrate 10.

The bio chip 20 includes an active surface 202 and a back surface 204. There is a plurality of I/O pads 22 which is arranged on two sides of the active surface 202 of the bio chip 20 and a connecting element 24 is disposed on each the plurality of I/O pads 22. A region (or can be named reaction zone) between the plurality of I/O pads 22 is used to implant with a plurality of biological probes 30, in which these biological probes 30 are used to capture the target object(s) in the detection solution, in which the target object(s) such as bacteria, viruses or suspended particle(s). It should be explained to that the formation of the bio chip 20 is carried out compatible with the existing semiconductor manufacturing process, and the formation method of the bio chip 20 is not a technical feature of this invention, so it is not to illustrate in detail herein.

Next, please continue to refer FIG. 1. The active surface 202 of the bio chip 20 is set toward to the bottom surface 104 of the substrate 10, and the reaction zone with a plurality of biological probes 30 on the active surface 20 of the bio chip 20 is aligned with the through hole 110 of the substrate 10. A connecting element 24 is arranged on each the plurality of I/O pads 22 on the active surface 202 of the bio chip 20 is electrically connected with the bottom surface 104 of the substrate 10, thereby, the volume of the entire bio chip package structure 1 may be narrowed. In some embodiments of this invention, the connecting element 24 is solder ball or solder plug and may electrically connect to the bottom surface 104 of the substrate 10 via the conductive glue (not shown).

There is a mold 40 arranged on the upper surface 102 of the substrate 10. The mold 40 having an opening 410 to align with the through hole 110 of the substrate 10, so the through hole 110 of the substrate 10 and opening 410 of the mold 40 exposes the plurality of biological probes 30 at the same time. It should be illustrated to that the aperture size of the through hole 110 is equivalent to the total reaction zone of the plurality of biological probes 30 on the active surface 202 of the bio chip 20. In addition, the mold 40 is flatly arranged on the upper surface 102 of the substrate 10 to solves the problem of setting the mold 40 that is necessary to consider the height difference between the bio chip 20 and substrate when the bio chip 20 and the substrate 10 and are electrically connected by wire bonding in the prior art, and the mold 40 cannot be sealed and fixed on the active surface 202 of the bio chip 20 with a sealant (not shown), which may make the detection solution (not shown) to overflow from the gap between the mold 40 and the bio chip 20 onto the substrate 10, and in addition to the inaccurate detection, the overflowing detection solution may also damage the entire bio chip package structure 1.

In addition to there is no height difference between the substrate 10 and the bio chip 20 when the mold 40 is only arranged on the upper surface 102 of the substrate 10, the mold 40 may seal and fix on the upper surface 102 of the substrate 10 with a sealant (not shown), so that the detection solution (not shown) is limited in the reaction zone of the plurality of biological probes 30 after the detection solution dropped, so as to the reaction zone of the plurality of biological probes can limit the random flow of the detection solution (not shown) to prevent the circuit layout (not shown) of the substrate 10 from contacting to the detection solution (not shown) to induce the short circuit of the bio chip package structure 1.

Another advantage of this invention is that the detection solution (not shown) can completely accommodate in the accommodating space which composed of the through hole 110 of the substrate 10 and the plurality of biological probes 30, so the volume of the detection solution is enough to be captured by the plurality of biological probes 30 of the bio chip 20, so the correctness and the precision of detection can be increased. In another embodiment of this invention, an upper cover (not shown) is also provided at the opening 410 of the mold 40. The purpose of the upper cover (not shown) is to prevent the bio chip package structure 1 before detecting process from the contamination entered into the plurality of biological probes 30 of the bio chip 20 via the opening 410 of the mold 40 to reduce the accuracy of the subsequent detecting process, in which the upper cover (not shown) can be to open or cover the opening 410 of mold 40 by sliding or page turning.

In addition, in some embodiment of this invention, a package structure 50 is provided to encapsulate the bottom surface 104 of the substrate 10 and the bio chip 20 except for the plurality of biological probes 30.

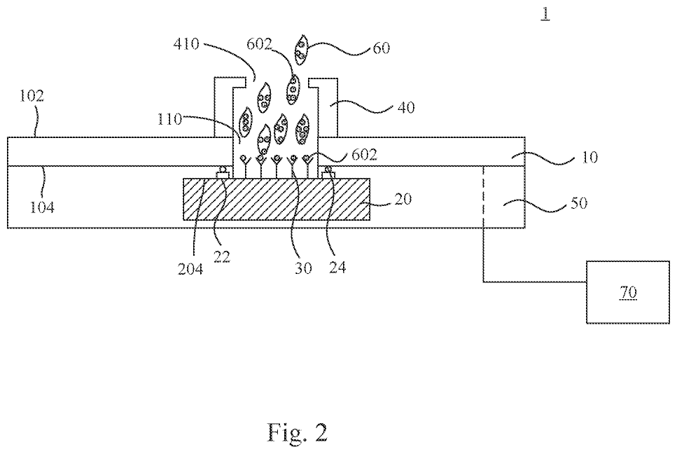

Please refer to FIG. 2. FIG. 2 is a schematic diagram of showing the detection of the detection solution by the bio chip package structure. In FIG. 2, the detection solution 60 with target object(s) 602 is dropped into the reaction zone formed by the plurality of biological probes 30 in the bio chip package structure 1. At this time, the detection solution 60 is accommodated in this space (reaction zone) formed by the mold 40, the through hole 110 of the substrate 10, and the plurality of biological probes 30, so that the plurality of biological probes 30 can capture the target object(s) in the detection solution 60 under a certain volume to increase the accuracy and the correctness of the detecting process. Meanwhile, the bio chip 20 also transmits the detection result through the substrate 10 to the socket (not shown) electrically connected with the substrate 10 or the external component 70 to obtain the result. In some preferred embodiments, the external component 70 may be a secure digital card (SD card) slot. It should be explained that the bio chip package structure 1 of this invention may arrange in a slot or electrically connect with a secure digital card slot, so the bio chip package structure 1 may portable and directly detect on site, and the result can obtain directly and quickly, and increase the efficiency and convenience of the detection.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A bio chip package structure, comprising:
a substrate, the substrate having a upper surface and a bottom surface, and a through hole passed through the upper surface and the bottom surface of the substrate;
a bio chip, the bio chip having an active surface and a back surface, a plurality of I/O pads is respectively disposed on two sides of the active surface of the bio chip, a plurality of biological probes arranged on a region between the plurality of I/O pads, and the active surface of the bio chip is set toward to make the plurality of biological probes that is aligned with the through hole of the substrate; and
a mold, the mold arranged on the upper surface of the substrate, the mold having an opening to align with the through hole of the substrate, so that the through hole of the substrate and the opening of the mold exposes the plurality of biological probes of the bio chip.

2. The bio chip package structure according to claim 1, wherein a connecting element is disposed on each the plurality of I/O pads on the active surface of the bio chip to electrical connect with the bottom surface of the substrate.

3. The bio chip package structure according to claim 2, wherein the connecting element is solder ball or solder plug.

4. The bio chip package structure according to claim 1, wherein the substrate is a printed circuit board with one or more layers of circuit layout.

5. The bio chip package structure according to claim 1, wherein a slot is disposed in the substrate.

6. The bio chip package structure according to claim 1, wherein an external component is electrically connected with the substrate.

7. The bio chip package structure according to claim 1, wherein the aperture size of the through hole of the substrate is identical to a total reaction zone of the plurality of biological probes on the active surface of the bio chip.

8. The bio chip package structure according to claim 1, further comprising a package structure to encapsulate the back side of the bio chip and the bottom surface of the substrate.

9. The bio chip package structure according to claim 1, wherein the opening of the mold is provided with a upper cover, and the upper cover opens or covers the opening of the mold by sliding or page turning.

10. The bio chip package structure according to claim 1, wherein the mold is fixed on the upper surface of the substrate with a sealant.
